(19) United States
(12) Patent Application Publication
(10) Pub. No.: US 2020/0000433 A1
(43) Pub. Date: Jan. 2, 2020

(54) ULTRASONOGRAPHIC METHOD FOR MEASURING MUSCLE DEFORMATION

(71) Applicants: Syddansk Universitet, Odense M (DK); Region Syddanmark, Vejle (DK)

(72) Inventors: Lars Henrik Frich, Odense M (DK); Kate Lykke Lambertsen, Odense SØ (DK); Jordi Sanchez Dahl, Odense C (DK); Anders Holsgaard Larsen, Odense SV (DK); John Hjarbæk, Odense SV (DK)

(21) Appl. No.: 16/482,550

(22) PCT Filed: Feb. 1, 2018
(86) PCT No.: PCT/DK2018/050021

§ 371 Date: Jul. 31, 2019

(30) Foreign Application Priority Data
Feb. 2, 2017 (EP) 17154342.4

(51) Int. Cl. A61B 8/08; A61B 5/0488

(52) U.S. Cl. CPC A61B 8/485; A61B 5/0488

(57) ABSTRACT

The invention relates to a method for determining deformation values of muscles, particularly skeletal muscles. According to the method, an image sequence of ultrasonic images of the muscle is obtained during a period where the patient performs a loaded action which causes the muscle to contract. During this period, the patient is guided to exert a predetermined force on an exercise device. The actually exerted force is measured simultaneously with the ultrasonic image sequence. Deformation values are determined based on the recorded image sequence. The invention also provides an apparatus for performing the method.

### FIELD OF THE INVENTION

The invention relates to ultrasonography methods, particularly to such methods for measuring deformation properties of muscles of the human body.

### BACKGROUND OF THE INVENTION

Muscle contraction is the activation of tension-generating sites within muscle fibers. Diseased, injured, or dysfunctional skeletal muscles may demonstrate abnormal function such as compromised contractility.

Treatment responses in muscle diseases may be evaluated on basis of evaluation of the muscle contractility. Such treatments may include medical treatments, e.g. medical treatment of patients with musculoskeletal (including sport injuries), neuromuscular diseases or physical rehabilitation of injured muscles. Therefore, evaluation of skeletal musculature contractility has important clinical applications.

Accordingly, there is a need for determining mechanical properties such as muscle deformation or muscle strain for the purpose of evaluating contractility of skeletal muscles. In order to be useful in clinical applications the method should enable reliable evaluation of muscle performance.

US 2016/0095575 discloses a method for quantitative dynamic evaluation of skeletal muscles functionality comprising the steps of a) receiving one or more sequences of two-dimensional or three-dimensional echographic images of the muscle under investigation b) transforming such sequence or sequences of images into sequences of measurements of deformations and/or strain rates in more spatial locations of the muscle or the muscles to evaluate c) outputting such sequences of spatial measurements in numeric and/or graphical format.

Whereas US 2016/0095575 shows a method for determining muscle strain a method which is more useful in clinical applications is needed.

### SUMMARY OF THE INVENTION

It would be advantageous to achieve an improved method for evaluating muscle performance, particularly for evaluating the parameters of contractility performance such as deformation capabilities. In particular, it may be seen as an object of the present invention to provide a method that solves the above mentioned problems relating to the reliability of clinical test methods, or other problems, of the prior art.

To better address one or more of these concerns, in a first aspect of the invention a method for determining deformation values indicative of contractility of a loaded muscle of a patient, the method comprises
setting a reference load to be exerted via a loaded action to be performed by the patient by use of the loaded muscle,
obtaining load values exerted by the patient during a measurement period comprising an action period in which the patient performs the loaded action,
based on the reference load and at least some of the obtained load values, guiding the patient performing the loaded action to exert the reference load using the loaded muscle during the action period,
obtaining an ultrasonography image sequence of the muscle at least during the action period,
selecting an analysis location in at least a part of the ultrasonography image sequence subsequent to the measurement period,
based on at least some of the ultrasonography images of the image sequence, determining the deformation values indicative of the contractility of the muscle as average deformation values based on determined displacements of different locations within the analysis location.

Advantageously, by guiding the patient to deliver a load corresponding to a desired reference force using the previously delivered load values during the action period the muscle load can be controlled. In this way the muscle deformation test can be performed under a desired load. For example, it may be desired that the load action is performed at a relatively low load due to the reduced capability of the injured or weakened muscle.

Furthermore, the deformation values from different tests can be compared since the tests are performed under corresponding load conditions. Accordingly, the patient can be instructed to perform the same test over time in order to assess changes in the determined deformation values over time, e.g. for the purpose of evaluating the effect of a treatment.

The obtained load values enables a clinician to assess the reliability of the determined deformation values, e.g. by checking if the obtained load values are close enough to the reference load.

US 2016/0095575 suggests using constrained kinematic machines and comparing determined deformations as a function of the type of the stress induced by the machine. Accordingly, US 2016/0095575 suggests a method enabling assessment of the effect of muscle deformation dependent on a type of the constrained kinematic motion. Since the constrained motion can be performed under different loads, US 2016/0095575 does achieve the advantage of enabling determination of deformation values for a given load or comparison of deformation values obtained over time for reliably evaluating the effect of a treatment.

The selection of the analysis location may be performed by selecting an analysis location in an image from the image sequence, e.g. an analysis location in the form of a selected area of that image. Thus, the selection of an analysis location in at least a part of the ultrasonography image sequence means a part such as an area having substantially the same location and coverage in each of a plurality of the images in the image sequence.

It is known to determine deformation values of muscles based on measured changes in muscle thickness. Compared to such methods the present method determines deformation values based on measured displacements of uniquely identifiable locations within a selected analysis location. Accordingly, the deformation values can be more accurate and are associated with a particular region in the muscle.

According to an embodiment, the analysis location is a single analysis location selected in at least a part of an image of the ultrasonography image sequence.

Advantageously, only a single analysis location is selected at a region within a muscle from where the deformation values are desired. Thus, the deformation values can be obtained so that they directly relate to the selected region. A small location, i.e. a small area, may be selected to obtain deformation values from a corresponding small region of the muscle. The analysis location may be selected as an area on an image, e.g. by drawing or otherwise selecting the desired area in an image of the image sequence.

According to an embodiment, each of the deformation values is associated with a specific zone in the analysis location which comprises at least one of the different locations.

Advantageously, the deformation values or the average deformation values are directly associated with the determined displacements of the different identifiable locations. Thus, the deformation values can be used for assessing the contractility of a narrowly defined zone, region or area of the muscle, in contrast to deformation values which characterizes the contractility of a non-specific area of a muscle.

According to an embodiment, the displacements are obtained based on tracking each of the different locations from image to image in the image sequence.

According to an embodiment, the different locations are different unique speckle patterns.

According to an embodiment, the image sequence is obtained at a rate greater than 100 images per second. For example, a framerate of 140 images per second may be used.

Advantageously, the high frame rate improves tracking and tracking accuracy of the different locations from image to image and thereby accuracy of the determined deformation values.

According to an embodiment, the method further comprises determining the deformation values associated with a deformation-direction, such as a predefined deformation-direction.

The deformation-direction may be a predefined or user defined direction along which displacements of locations, e.g. speckle patterns, are determined. Advantageously, the deformation values have a well-defined direction which is a direction of displacements of the different locations. Since the deformation values are obtained directly on basis of displacement of specific regions in the muscle, the displacement direction, or a projection thereof along a defined direction, is directly correlated with the deformation direction or is identical with the deformation direction. Advantageously, since the deformation direction is known or can be determined, the deformation values can be determined for a particular direction.

Furthermore, the deformation values can be determined for two or more deformation-directions. The deformation values for the one or more deformation-directions can be are determined based on a direction of displacements of at least one of the locations. That is, when the direction of displacements are known, the deformation values along other directions can be obtained, e.g. based on projection of the determined displacements along a direction of interest.

According to an embodiment the setting of the reference load to be exerted comprises setting the reference load to a percentage of a measured maximum load exerted via the loaded action previously performed by the patient.

Advantageously, by providing a function for setting the reference to a percentage of the maximum load, the deformation test can be performed at different load levels for achieving a better characterization of the contractility.

According to an embodiment, the maximum load can be obtained according to a method where, prior to setting the reference load, peak load values exerted by the patient are obtained during a peak load action period in which the patient exerts a maximum load while performing the loaded action, and where the measured maximum load are determined from the obtained peak load values. For example, the measured maximum load can be obtained as an average of the obtained peak load values. Accordingly, by providing a function for obtaining and e.g. storing the peak load values and determining the maximum load it becomes possible to evaluate the contractility at well-defined load levels below the maximum load.

For example, the function for obtaining the maximum load may be arranged to instruct the patient to exert the maximum load during two or more peak load action periods, where a subsequent action period is separated from the previous one by a relaxation period. After the load values from the loaded actions have been obtained, the maximum load can be determined from one or more of the peak load action periods.

According to an embodiment the guiding of the patient comprises displaying the reference load and a history of at least some of the load values obtained during the action period. The presentation of the reference together with the measured loads, e.g. on a display, may advantageously provide a visual feedback enabling the patient to adjust the exerted load towards the reference load.

Due to the functionality of the first aspect enabling determination of deformation values dependent on a reference load, an embodiment of the invention is particularly suited for determining deformation values of skeletal muscles.

According to an embodiment the loaded action is performed by exerting a load on an exercise machine capable of measuring the exerted load. The measurement of the load may be performed by known methods, e.g. by use of a potentiometer coupled with a spring loaded measurement system.

According to an embodiment the measurement period is initiated by a user activated trigger function. Advantageously, the functionality may include a function for starting the measurement period and possibly for ending the measurement period. Accordingly, by use of a trigger function the obtained load values and the ultrasonography image sequence can be synchronized in time. Furthermore, by defining the length of the measurement period by both initiating and ending the measurement period by the trigger function, the length of the measurement period can be determined in advance.

According to an embodiment the method further comprises selecting an analysis location in at least a part of the ultrasonography image sequence subsequent to the measurement period, where the determination of the deformation values is based on the selected analysis location in a plurality of the ultrasonography images in the ultrasonography image sequence.

According to an embodiment each of deformation values are determined on basis of at least two of the ultrasonography images in the ultrasonography image sequence, where each of the at least two ultrasonography images have been obtained at different time points during the action period.

According to an embodiment the method further comprises obtaining an EMG signal indicative of the myoelectric activity of the muscle at least during the action period. Advantageously, since the EMG signal should correlate in time with the load values, the EMG signal can be used, in addition to the obtained load values, to verify the obtained deformation values. For example, according to an embodiment, the functionality may include displaying the deformation values and the EMG signal at least for the action period, where the displaying is performed subsequent to the measurement period. Additionally or alternatively, the functionality may include displaying the deformation values and the force values at least for the action period, where the displaying is performed subsequent to the measurement period.

According to an embodiment, the process of obtaining the load values and the ultrasonography image sequence may be synchronized in time. Additionally, the EMG signal may be obtained so that the EMG signal is also synchronized in time at least with the ultrasonography image sequence. In this way, the deformation values determined from the ultrasonography image sequence can be displayed in time synchronization with the load values and/or the EMG signal in order to enable a direct comparison for verification of the deformation values.

A second aspect of the invention relates to a processing device arranged for obtaining an ultrasonography image sequence for determining deformation values indicative of contractility of a loaded muscle of a patient, the device comprises
a guiding function arranged to receive,
   a reference load to be exerted via a loaded action to be performed by the patient by use of the loaded muscle,
   load values exerted by the patient during a measurement period comprising an action period in which the patient performs the loaded action, where the guiding function is further arranged to guide the patient performing the loaded action to exert the reference load using the loaded muscle based on the reference load and at least some of the obtained load values, and
an analyzing function arranged to receive the ultrasonography image sequence of the muscle, where the ultrasonography image sequence is obtained at least for the action period, and arranged to determine the deformation values indicative of the contractility of the muscle based on at least some of the ultrasonography images of the image sequence.

The processing device may be configured so that the guiding function of the processing device is active in data acquisition period where load data and ultrasound video data are obtained. The analyzing function may comprise a video recording function for receiving and storing the ultrasonography image sequence during the data acquisition period and a processing function for processing the image sequence for determination of the deformation values during an analyzing period subsequent to the data acquisition period. It is understood that the processing device could consist of a single device or two or more separate units, e.g. a unit for the guiding function and the video recording function and another unit for the processing function.

A third aspect of the invention relates to an ultrasonic video recording and processing system arranged for obtaining an ultrasonography image sequence for determining deformation values indicative of contractility of a loaded muscle of a patient, where the system comprises
the processing device according to the second aspect, and
an ultrasound scanner arranged for obtaining the ultrasonography image sequence.

Accordingly, one or more of functions of various embodiments may be comprised by ultrasonic video recording and processing system, e.g. a system dedicated for obtaining and determining deformation values of a loaded muscle such as skeletal muscles.

A further aspect of the invention relates to computer program product directly loadable into an internal memory of a digital computer where the computer program product comprises software code portions for performing the steps of the method according to the first aspect when the computer program product is run on the computer.

In general the various aspects of the invention may be combined and coupled in any way possible within the scope of the invention. These and other aspects, features and/or advantages of the invention will be apparent from and elucidated with reference to the embodiments described hereinafter.

### BRIEF DESCRIPTION OF THE DRAWINGS

Embodiments of the invention will be described, by way of example only, with reference to the drawings, in which

FIG. 1 shows a processing device configured to determine deformation values of a loaded muscle,

FIG. 2 illustrates the reference load, the obtained load values, the determined deformation values and the EMG signal for illustration of the principle of an embodiment of the invention,

FIG. 3 illustrates an example of a user interface of the processing device for displaying results from the scanning procedure,

FIG. 4 shows an example of load values determined for different load percentages and correlation between strain and muscle load,

FIGS. 5A-B illustrate methods for determining the deformation values from tracked displacements of identifiable locations or local identifiers, and

FIG. 6 shows another example of experimentally obtained mean strain values for different load percentages.

### DETAILED DESCRIPTION OF EMBODIMENT

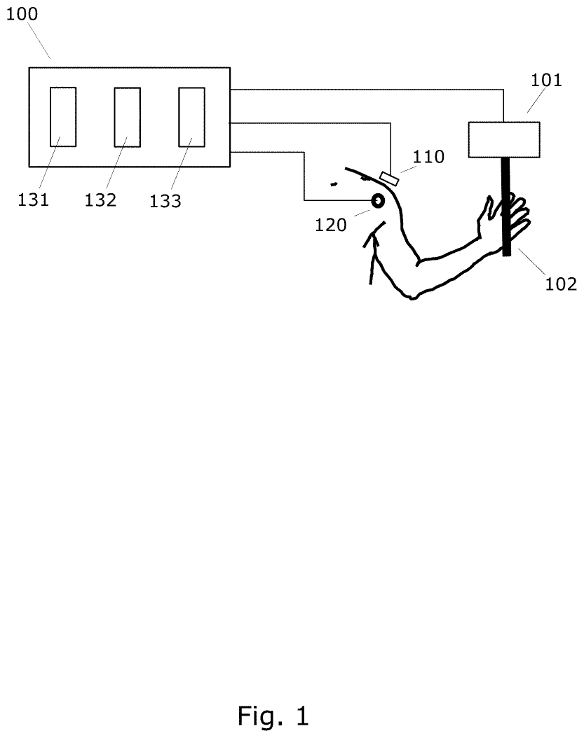

FIG. 1 shows a processing device 100 configured to obtain an ultrasonography image sequence and determining deformation values indicative of contractility of a loaded muscle of a patient based on the image sequence. The deformation values may be in the form of strain values or other related mechanical property values of the muscle.

The processing device 100 may be an electronic device comprising electronic circuits, digital processors or a combination thereof arranged for carrying out the functions of the image processing device 100. A computer program arranged for performing at least some functions of the processing device 100 may be directly loadable into an internal memory of the processing device 100 or may be stored in an internal memory of the processing device 100.

The processing device 100 is arranged to receive load values from an exercise machine 101. The exercise machine 101 measures the load exerted by a patient on a load receiving input 102.

For example, as illustrated, the exercise machine 101 may be arranged to load muscles of an arm of a patient when the patient performs a loaded activity facilitated by the exercise machine 101, e.g. a movement of a string loaded input handle 102. The load values may be in the form of force, torque, power, pressure or other equivalent values relating to the force delivered by the patient on the exercise machine. The exercise machine is arranged to enable the patient to load the machine with different loads, e.g. loads from a zero load up to the maximal load that the patient is able to produce. The exercise machine has load sensor, e.g. a force sensor or other measurement unit, arranged to measure the applied load and arranged to make the measurements available for the processing device 100.

The loaded activity performed by the user loads the muscle under investigation and thereby causes muscle contraction.

The processing device 100 is arranged to receive and process ultrasonography image sequences from an ultrasound scanner 110. The ultrasound scanner 110 is used for obtaining the image sequences of the muscle used under investigation.

The ultrasound scanner 110 comprises a probe, e.g. a hand held scanner probe. Examples of the probe comprise linear probes such as a 11L-D probe, convex probes and other array or non-array probes. The ultrasound scanner 110 further comprises electric circuits for driving and receiving electronic signals from the scanner probe. The electric circuits of the ultrasound scanner may be comprised by the processing device 100 or may be comprised by the scanner 110 or other external device.

The processing device 100 may further be arranged to receive EMG (Electromyography) signals from an EMG sensor 120. The EMG sensor is used for measuring the myoelectric activity produced by the muscle under investigation.

The processing device 100 comprises a guiding function 131 arranged to obtain a reference load to be exerted via the loaded action performed by the patient. The reference load may be determined by the processing device 100. For example, the reference load may be determined on basis of a peak load value previously exerted by the patient in connection with a peak load test, the reference load may be received as a signal via an input of the image processing device 100 or the reference load may be entered via a user interface of the image processing device 100.

The guiding function 131 is configured to guide the patient to exert the reference load using the loaded muscle when he performs the loaded action.

The processing device 100 further comprises an analyzing function 132 arranged to receive and process ultrasonography image sequences from the ultrasound scanner 110.

The processing device 100 is particularly configured to determine contractility of skeletal muscles such as a muscles of the thighbone, rotator cuff muscles or other. In order to obtain useful image sequences, the ultrasonic scanner 110 is configured to obtain the image sequences at a relevant tissue depth, e.g. depths within a range from 2 to 8 cm. The scanner probe may be operated a frequency within a range from 7-18 MHz. Preferably, the scanner probe is a linear scanner probe.

An aspect of the invention relates to an ultrasonic video recording and processing system which in addition to the processing device 100 comprises the ultrasound scanner 110, e.g. the electric circuit for driving and receiving electronic signals from the scanner probe, possibly other electric circuits for driving and processing signals from the exercise machine 101 and/or the EMG sensor 120.

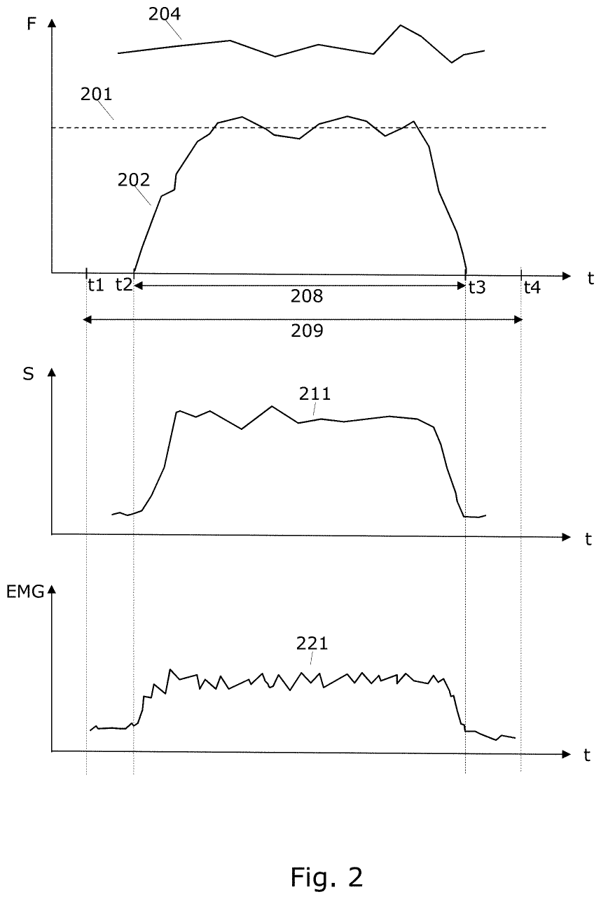

FIG. 2 together with FIG. 1, illustrates the principle of an embodiment of the invention. The coordinate systems show the load (F), the deformation (S) and EMG amplitude (EMG) as a function of time (t).

The reference load 201 is set in order to guide the patient to deliver a load close to the reference load. For example, the reference load 201 may be displayed together with the actually delivered load values 202 received from the exercise machine 101. Alternatively, the reference load 201 may be used to generate a guiding sound informing the patient if the delivered load is within an acceptable range, too high or too low.

Accordingly, the guiding of the patient to exert the reference load may be based on the reference load 201 and at least some of the obtained load values 202. For example, the received load values may low-pass filtered, e.g. on basis of a fixed number of load values received most recently, in order to guide the patient based on the most recently and smoothed values of the delivered load.

The measurement period 209 defines a period wherein the processing device 100 stores or samples the received load values 202, the ultrasonography image sequence and optionally the EMG values 221. The action period 208 is a sub-period of the measurement period 209 and defines the period where the patient performs the loaded action.

The start of the measurement period 209 at t1 may be initiated by a user activated trigger function. The user activated trigger function may be an external trigger device which upon activation sends a trigger signal to the processing device 100, or the user activated trigger function may be embodied by a user interface, e.g. a start button, of the processing device 100.

The end of the measurement period at t4 may be determined via the same trigger function, e.g. a stop button of the processing device 100, e.g. by a timer function which stops the measurement after a predetermined period of time after the start of the measurement period 209. Accordingly, by means of the start and stop functions of the trigger function the length of the measurement period can be adapted to the load activity. Furthermore, repeated contractility tests of a given patient can be performed with equal lengths of the measurement period 209.

The reference load 201 may be obtained as a percentage of a measured maximum load 204 exerted via the loaded action previously performed by the patient. FIG. 2 principally illustrates a maximum load curve 204 which has been obtained at an earlier time. The maximum load values 204 may have been obtained in the same way as the load values 202 by use of the exercise machine 101 and the processing device 100, but instead of guiding the patient to deliver the reference load 201 the patient is instructed to deliver the highest possible load. For example, the processing device 100 may initiate and end one or more measurement periods separated by relaxation periods in which the patient is instructed to exert a maximum force.

Accordingly, the processing device 100 may be configured to obtain peak load values exerted by the patient during a peak load action period, or two or more peak load action periods, in which the patient exerts a maximum load while performing the loaded action, and to determine the measured maximum load 204 from the obtained peak load values. The peak load action period may correspond to the action period 208 and, therefore, the measurement period 209 may be used for receiving and storing the peak load values. The processing device 100 may be provided with a peak force function which may comprise a trigger function which starts storing load values from the exercise machine 101 and which may disable the guiding function 131.

During at least the action period 208, e.g. during a period which starts before the start of the action period 208 at time t2 and which ends after the end of the action period 208 at time t3, the ultrasonography image sequences are received and stored by the processing device 100.

Similarly, during at least the action period 208 or during the entire measurement period 209 the processing device 100 (e.g. the EMG function 133), in an embodiment, receives and stores the EMG signal 221.

The processing device 100 is arranged to display one or more of the received load signals 202, the determined deformation values 211 and the EMG signal 221.

After completion of the measurement period 209, the processing device 100 determines deformation values of the loaded muscle by processing of the ultrasonography image sequence. Thus, processing of the stored ultrasonography image sequence for determination of deformation values can be performed at any time after the end of the measurement period 209.

The deformation values are determined on basis of at least two of the ultrasonography images of the image sequence obtained at different time points during the action period.

Deformation values may be determined by known methods. For example, the speckle pattern which inherently is present in the received ultrasonography image sequence can be used to determine contractions or elongations in the muscle by analyzing speckle patterns in the sequence of ultrasonography images obtained at different time points. The speckle pattern is created by interference of the scattered reflections from structures and cells of muscle and creates a random spatial speckle pattern. Accordingly, any location of the imaged muscle has a unique speckle pattern which can be tracked over image frames of the image sequence. Accordingly, the deformation for a given area or volume of the muscle can be determined by determining changes in a distance between uniquely identified local speckle patterns in an image from image frame to image frame.

By use of image analysis, other uniquely identified local markers in the image frames of the ultrasonography image sequence can be traced from frame to frame in order to determine changes in distance between such markers.

Accordingly, by determining changes in distances between particular local identifiers in the ultrasonography image sequence between consecutive image frames the deformation values or other relates mechanical property values (e.g. strain values) indicative of the contractility of the muscle can be determined. It is not necessary to use all received and stored images, e.g. if the frame rate is high. Therefore, the deformation values are determined based on at least some of the ultrasonography image frames of the image sequence.

FIG. 2 shows that the determined deformation values 211 may be displayed together with the EMG signal 221 or the force values 202 or both. The values and signals may be displayed at least for the action period 208, possibly for the entire measurement period 209. Since the deformation values 211 are determined subsequent to the measurement period 209, the presentation of the values and signals on a display may be performed subsequent to the measurement period.

The processing device 100 receives and stores the load values 202, the ultrasonography image sequence and the optional EMG signal 221 in a synchronized way, i.e. so that the stores values are synchronized in time. Accordingly, a value of the deformation curve 211 at a given point in time can be directly compared with a value of the load curve 202 and/or the EMG signal 221.

The presentation of the deformation curve 211 together with load curve and/or the EMG signal 221 enables a clinician or other user of the processing device 100 to assess the validity of the determined strain curve 211, e.g. by assessing if the strain values 211 start increasing at the same time as the force values 202 and/or the values of the EMG signal 221.

The deformation curve 211 can be used to assess the contractility of the muscle under investigation. For example, deformation values below expected values for a given level of the load values 202 indicates low contractility. A low contractility could indicate low responsivity to a medical treatment or could indicate that that an injured muscle has not jet rehabilitated sufficiently.

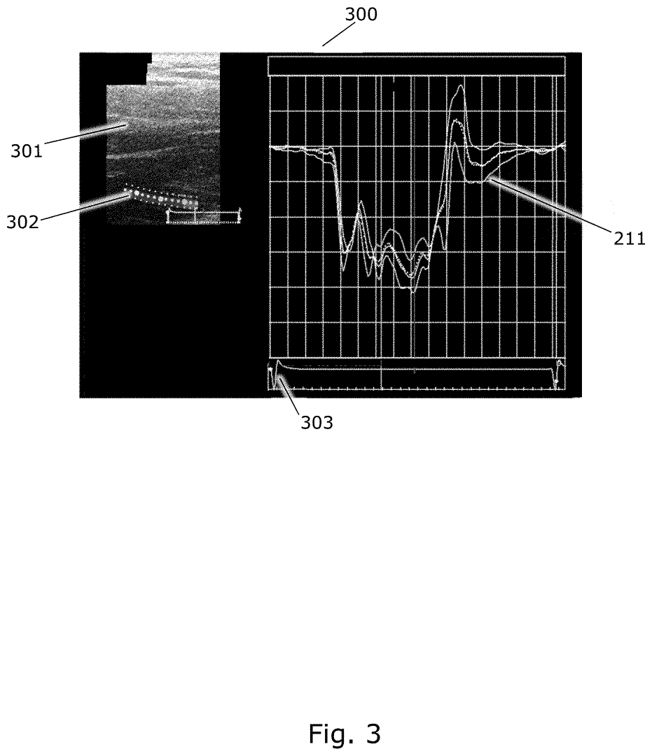

FIG. 3 shows an example of a user interface 300 of the processing device 100 for displaying results from the scanning procedure. The user interface shows the determined deformation values 211. In this example, the deformation values are determined for different locations of the muscle so that three different deformation curves are shown. FIG. 3 also shows a trigger signal 303 for starting ending the measurement period 209.

The user interface 300 also shows a video presenter 301 arranged for displaying the received ultrasonography image sequence. The image in the video presenter 301 shows a cross section of the muscle. In practice the video presenter 301 shows the received ultrasonography image sequence or a part of it, e.g. a shortened image sequence, in order to show the location and direction of muscle contractions.

The video presenter 301 may be used for selecting an analysis location 302 in the ultrasonography image sequence. The selection of the analysis location 302 may be performed manually or the selection may be assisted by a selection function of the processing device 100 or performed automatically based on image analysis.

The selected analysis location 302 determines the location (e.g. location of an area, point, a line or other shape) in the ultrasonography image sequence obtained during the measurement period 209 from which the determination of the deformation values should be based. The user interface 300 may be configured to select one or more analysis locations 302 for determination of corresponding one or more deformation curves 211.

As an example, the deformation values determined for the selected analysis location 302 could be determined as an average of deformation values obtained at different locations within the analysis location 302, as deformation values obtained at a location within the analysis location 302 having, e.g. in average, the smallest or greatest deformation values. The distance between the local identifiers, e.g. unique speckle patterns, in the ultrasonography image sequence could correspond to a dimension, e.g. a length, of the selected analysis location 302, a fraction of the dimension, or a distance corresponding to a minimum distance between arbitrarily selected unique local identifiers within the selected analysis location 302 or in the vicinity of the selected analysis location 302 (e.g. if the location 302 is selected as a point).

Thus, by tracking identifiable locations such as unique speckle patterns, within a selected analysis location 302 in an image of the image sequence from image to image, the deformation values 211 can be determined from tracked displacements of at least one identifiable location, preferably two or more different identifiable locations. By use of two or more identifiable locations, average deformations, i.e. average deformation values as a function of time, can be obtained.

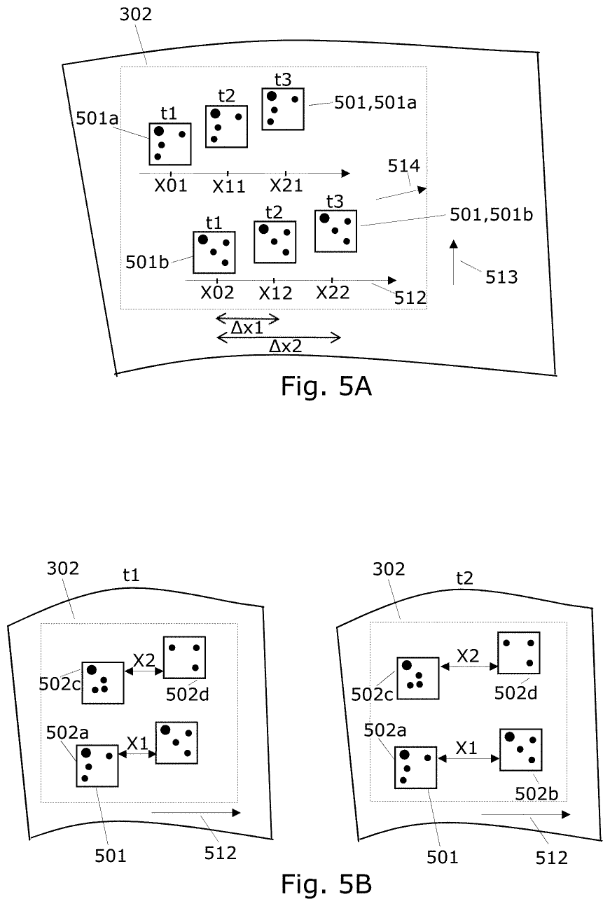

FIGS. 5A-B illustrate methods for determining the deformation values 211 from tracked displacements of identifiable locations 501, i.e. local identifiers, in a selected analysis location 302, from images with different recording times t1, t2, t3, in the image sequence.

FIG. 5A shows a first uniquely identifiable location 501a. The uniquely identifiable location 501a, e.g. a unique speckle pattern or other traceable image pattern, is identified at time t1, e.g. in the same image as the analysis location 302 is identified. The location 501a is tracked in successive images recorded at times t1-t3. The locations 501 may be identified and traced by automatic image analysis methods. The positions x01, x11, x21 of the locations 501a relative to a stationary reference location, alternatively coordinates x01, x11, x21 along the displacement direction 502, is determined, e.g. from a determined center of the locations 501. It is understood that the locations 501 has a two-dimensional extension in the image plane of the images, e.g. extensions in the range from 1 mm to 30 mm.

Displacements of the locations 501 as a function of time can be determined from the positions x01, x11, x21 obtained at different times t1-t3, e.g. as a displacement Δx1(t2)=x11−x01 and Δx1(t3)=x21−x01 and so forth. The deformation values 211 may be identical with the displacements Δx1 or they may be determined as a relative displacement, e.g. as a strain value, on basis of the positions x01, x11, x21. For example, relative displacements could be obtained as ratio of Δx1(t) and L, where L may be a predetermined length, a dimension of the analysis location 302, or other.

Similarly, based on a second uniquely identifiable location 501b, positions x02, x12, x22, corresponding displacements Δx2(t2)=x12-x02 and Δx2(t3)=x22-x02 and deformation values 211 can be determined.

When two or more uniquely identifiable locations 501a, 501b are used for determining deformation values 211, the deformation values 211 can be determined as average deformation values 211 based on displacements Δx1, Δx2. For example, the average of the displacements Δx1, Δx2 can be determined as average displacement as a function of time, and/or an average deformation 211 as a function of time can be determined from displacements Δx1(t), Δx2(t).

FIG. 5B shows an alternative method for determining deformation values 211 on basis of at least two locations 502a, 502b, e.g. on basis of one or more distances X1, X2 between corresponding one or more pairs of locations 502a, 502b at different recording times t1, t2 of the images in the image sequence. The displacements such as Δx1(t2)=X1(t2)−X1(t1) can be determined from a single pair of locations 502a, 502b for successive points in time. Advantageously, two or more displacements, e.g. Δx2(t2)=X2(t2)−X2(t1) and Δx1(t2), is determined from corresponding two or more pairs of locations 502a, 502b and 502c, 502d as illustrated. Deformation values 211 can be determined as Δx/Δx0 where Δx0 may be an initial distance between a pair of locations 502a, 502b. Average deformation values 211 can be determined based on at least four different locations 502a, 502b, e.g. by averaging deformation values 211 determined from two or more pairs of locations 502a-d.

Since the deformation values 211 are determined directly from displacements of the locations 501, the deformation-direction associated with the deformation values 211 can be determined based on a direction 514 of the displacements of the locations 501. The direction 514 can be projected onto directions of interest such as perpendicular directions 512, 513, e.g. directions 512, 513 from which the deformations 211 are determined.

The method may further comprise selecting the directions 512, 513 from at least two different directions, e.g. perpendicular directions in the plane of an image or an arbitrarily defined direction in the image. Since different displacement directions may be of interest for different purposes, a particular direction such the direction 512 or a perpendicular direction may be selected in order to obtain deformation values 211 associated with the selected direction. Thus, the one or more of the directions of interest 512, 513 may be predefined such as user defined. Similarly, the method may comprise determining the deformation values 211 for one or more directions, e.g. predefined directions, such as deformation values along a main displacement direction 512 along which displacement mainly takes place and an associated perpendicular direction 513.

Thus, the deformation values 211 associated with one or more deformation-directions can be determined based on displacements Δx determined along one of the directions of interest 512, 513, 514, or by projection of main displacement values along a main direction 514 onto one or more directions of interest 512, 513. Clearly, the direction of interest can be the main direction of displacement 514.

Since the deformation values 211 are determined directly from displacements Δx of locations 501 within the analysis location 302 a single selected analysis location is sufficient. The analysis location 302 may be selected in a part of the ultrasonography image so that the image contained in the analysis location 302 does not comprise boundaries of the muscle, or at least so that the locations 501 does not comprise muscle boundaries, like skin and bone boundaries.

Alternatively, the image contained in the analysis location 302 may comprise muscle boundaries. This could be used as an identifier for subsequent ultrasound scanning procedures, e.g. to monitor an effect of a treatment over time, so that the deformation values 211 can be determined for the same muscle location.

Since the tracking of the identifiable locations 501 is based on tracking of fine structures of the muscle or tracking of speckle patterns a sufficient high frame rate of the image sequence is required. That is, since the tracking is not based on image content containing easily identifiable structures like boundaries of the muscle, a relative high frame rate is required. Thus, the image sequence may be obtained at a rate greater than 100 images per second, e.g. within a range from 120 to 180 such as 140 images per second.

Advantageously, since the image does not comprise parts which may be influenced by pressures on the skin, e.g. pressures originating from the scanner probe, more reliable results can be obtained.

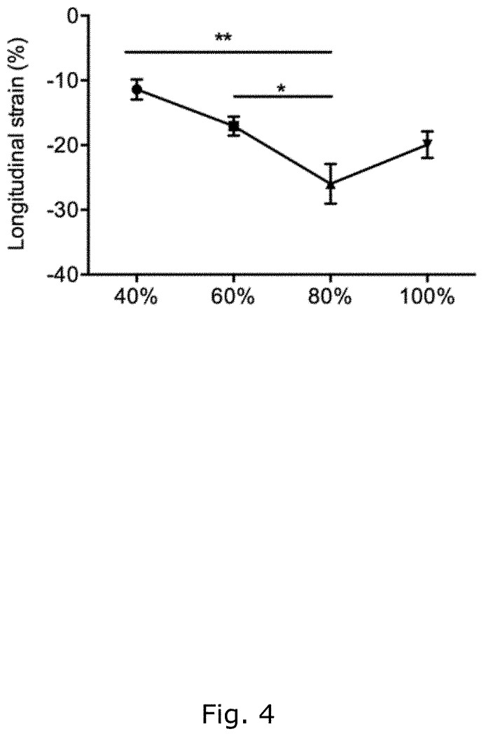

FIG. 4 shows an example of strain values determined for different load percentages. The graph shows an expected increase of the strain as the load is increased from 40% to 80% of the maximum load. At 100% load, the strain valued would be expected to be even higher. The illustrated reduction in the strain at 100% load is incorrect and is due to variations in the force delivered by the patient during the measurement period. This also indicates the necessity of performing the muscle deformation test at loads below the maximum load.

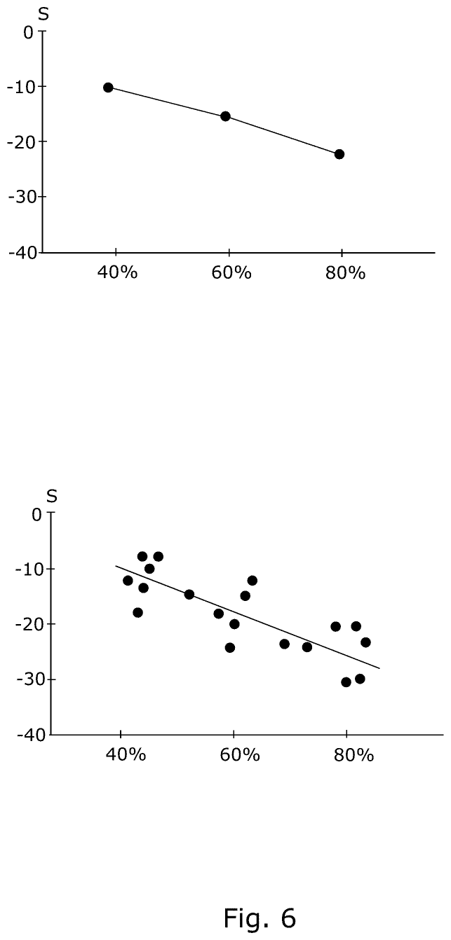

FIG. 6 shows another example of experimentally obtained mean strain values S for different load percentages (from 40% to 80% of a maximum 100% load). FIG. 6 largely corresponds with FIG. 4 for the interval from 40-80%, but has been obtained based on other experiments. The mean strain values which correspond with deformation values 211 are obtained for the normal supraspinatus muscle of ten subjects subjected to increasing isometric loads from 40% to 80% relative to a maximum load of 100% obtained for each subject. The upper graph shows an almost linear relationship between load and deformation which shows that the method is reliable and capable of determining strain values over a large range of loads. The lower graph shows that the experimentally obtained measurements from the ten subjects are highly correlated.

Although the present invention has been described in connection with the specified embodiments, it should not be construed as being in any way limited to the presented examples. The scope of the present invention is to be interpreted in the light of the accompanying claim set. In the context of the claims, the terms “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

1-23. (canceled)

24. A method for determining strain values indicative of contractility of a loaded muscle of a patient, the method comprises
setting a reference load to be exerted via a loaded action to be performed by the patient by use of the loaded muscle,
obtaining load values exerted by the patient during a measurement period comprising an action period in which the patient performs the loaded action,
based on the reference load and at least some of the obtained load values, guiding the patient performing the loaded action to exert the reference load using the loaded muscle during the action period,
obtaining an ultrasonography image sequence of the muscle at least during the action period,
selecting an analysis location (302) in at least a part of the ultrasonography image sequence subsequent to the measurement period,
based on at least some of the ultrasonography images of the image sequence, determining the strain values (211) indicative of the contractility of the muscle based on determined displacements (Δx1(t2), Δx1(t2))) of at least one identifiable location (501) within the analysis location (302), where the displacements (Δx1(t2), Δx1(t2)) are obtained based on tracking the at least one identifiable location (501) from image to image in the image sequence.

25. A method according to claim 24, where the analysis location (302) is a single analysis location selected in at least a part of an image of the ultrasonography image sequence.

26. A method according to claim 24, where each of the deformation values (211) are associated with a specific zone in the analysis location 302 which comprises at least one of the different locations (501).

27. A method according to claim 24, where the displacements are obtained based on tracking each of the different locations (501) from image to image in the image sequence.

28. A method according to claim 24, where the different locations (501) are different unique speckle patterns.

29. A method according to claim 24, where the image sequence is obtained at a rate greater than 100 images per second.

30. A method according to claim 24, further comprising determining the deformation values (211) associated with a predefined deformation-direction (512, 513, 514).

31. A method according to claim 30, comprising determining the deformation values (211) for two or more predefined deformation-directions (512, 513, 514).

32. A method according to claim 30, where the deformation values (211) for the one or more predefined deformation-directions (512, 513, 514) are determined based on a direction (514) of displacements of at least one of the locations (501).

33. A method according to claim 24, where setting the reference load to be exerted comprises setting the reference load to a percentage of a measured maximum load exerted via the loaded action previously performed by the patient.

34. A method according to claim 33, further comprising
prior to setting the reference load, obtaining peak load values exerted by the patient during a peak load action period in which the patient exerts a maximum load while performing the loaded action,
determining the measured maximum load from the obtained peak load values.

35. A method according to claim 24, where guiding the patient comprises displaying the reference load and a history of at least some of the load values obtained during the action period.

36. A method according to claim 24, where the muscle is a skeletal muscle.

37. A method according to claim 24, where the loaded action is performed by exerting a load on an exercise machine capable of measuring the exerted load.

38. A method according to claim 24, where the measurement period is initiated by a user activated trigger function.

39. A method according to claim 24, where the method further comprises selecting an analysis location in at least a part of the ultrasonography image sequence subsequent to the measurement period, and where the determination of the deformation values is based on the selected analysis location in a plurality of the ultrasonography images in the ultrasonography image sequence.

40. A method according to claim 24, where each of deformation values are determined on basis of at least two of the ultrasonography images in the ultrasonography image sequence, where each of the at least two ultrasonography images have been obtained at different time points during the action period.

41. A method according to claim 24, where the method further comprises obtaining an EMG signal indicative of the myoelectric activity of the muscle at least during the action period.

42. A method according to claim 41, where the method comprises displaying the deformation values and the EMG signal at least for the action period, where the displaying is performed subsequent to the measurement period.

43. A method according to claim 24, where the method comprises displaying the deformation values and the force values at least for the action period, where the displaying is performed subsequent to the measurement period.

44. A method according to claim 24, where the process of obtaining the load values and the ultrasonography image sequence are synchronized in time.

45. A processing device arranged for obtaining an ultrasonography image sequence for determining strain values indicative of contractility of a loaded muscle of a patient, the device comprises
a guiding function arranged to receive,
a reference load to be exerted via a loaded action to be performed by the patient by use of the loaded muscle,
load values exerted by the patient during a measurement period comprising an action period in which the patient performs the loaded action, where the guiding function is further arranged to guide the patient performing the loaded action to exert the reference load using the loaded muscle based on the reference load and at least some of the obtained load values, and
an analyzing function arranged to receive the ultrasonography image sequence of the muscle, where the ultrasonography image sequence is obtained at least for the action period, and arranged to determine the strain values (211) indicative of the contractility of the muscle based on at least some of the ultrasonography images of the image sequence and based on determined displacements (Δx1(t2), Δx1(t2)) of at least one identifiable location (501) within an analysis location (302) selected in at least a part of an image of the ultrasonography image sequence, where the displacements (Δx1(t2), Δx1(t2)) are obtained based on tracking the at least one identifiable location (501) from image to image in the image sequence.

46. An ultrasonic video recording and processing system arranged for obtaining an ultrasonography image sequence for determining deformation values indicative of contractility of a loaded muscle of a patient, the system comprises
the ultrasonic image processing device according to claim 45, and
an ultrasound scanner arranged for obtaining the ultrasonography image sequence.
